(19) United States
(12) Patent Application Publication
(10) Pub. No.: US 2002/0072602 A1
(43) Pub. Date: Jun. 13, 2002

(54) Micronized mirtazapine

(76) Inventor: Claude Singer, Kafar Saba (IL)

Correspondence Address: KENYON & KENYON, ONE BROADWAY, NEW YORK, NY, 10004, US

(21) Appl. No.: 09/901,271

(22) Filed: Jul. 10, 2001

Related U.S. Application Data
(60) Provisional application No. 60/216,564, filed on Jul. 7, 2000.

(51) Int. Cl. C07D 487/22; B02C 17/02

(52) U.S. Cl. 540/555; 241/24.1

(57) ABSTRACT

The present invention relates to mirtazapine having relatively small particles, and corresponding relatively large surface area. In one embodiment, the invention relates to mirtazapine and formulations containing mirtazapine having a mean particle diameter of less than 200 micrometer,

### CROSS-REFERENCE TO RELATED APPLICATION

The present application claims benefit of U.S provisional application Ser. No. 60/216,564, filed Jul. 7, 2000, which is incorporated by reference.

### FIELD OF THE INVENTION

This invention relates to micronized mirtazapine and to the preparation thereof.

### BACKGROUND OF THE INVENTION

Mirtazapine, 1,2,3,4,10,14b-hexahydro-2-methyl-pyrazino [2,1 -a]pyrido[2,3-c][2] benzazepine, having the formula I:

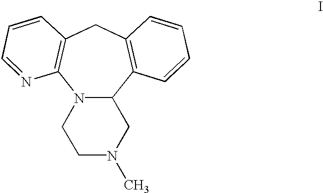

is approved, under the trademark Remeron®, by the U.S. Food and Drug Administration, for the treatment of depression. Mirtazapine has a tetracyclic chemical structure unrelated to other classes of antidepressants such as selective serotonin reuptake inhibitors, tricyclics or monoamine oxidase inhibitors.

U.S. Pat. No. 4,062,848, the contents of which is incorporated by reference, discloses a process for making mirtazapine.

Mirtazapine is essentially insoluble in water. The Particle Size Distribution (PSD) of mirtazapine crystals may be used to determine the available surface area for the drug dissolution thus effecting the solubility. Often, it is observed that the available surface area for drug dissolution correlates to the rate of dissolution and solubility where a greater surface area enhances the solubility f a drug and enhances the rate of dissolution of a drug. Further, the velocity of dissolution of a drug often effects the drug's bioavailability. Thus the PSD of mirtazapine and, in particular, the mean particle diameter are important parameters to characterize and predict the bioavailability of the drug. It is desireable to have mirtazapine with a particle size in which the mean particle size enhances the reporducability of the rat of dissolution and the reproducibility of the dissolution. It is desirable to have mirtazapine in which the mean particle size imparts an improved and stable dissolution profile.

### SUMMARY OF THE INVENTION

The present invention relates to mirtazapine and mirtazapine formulations containing mirtazapine having relatively small particles, and corresponding relatively large surface area.

In one embodiment, the invention relates to mirtazapine having a mean particle diameter of less than 200 micrometer, preferably mean particle diameter is less than 100 microns, more preferably the mean particle diameter is less than 20 microns, most preferably the mean particle size is between about 10 and 20 microns. Micronized mirtazapine is used herein as referring to mirtazapine having a mean particle diameter of about 200 to about 10 microns.

According to another embodiment of the invention, the present invention relates to a process for preparing micronized mirtazapine. Mirtazapine, prepared by methods known the art, is separated by sieves to produce mirtazapine having a mean particle diameter of about 350 to about 250 microns. Preferably the mirtazapine has a mean particle diameter of about 300 to 250 microns. The sieved mirtazapine is then micronized in a micronized to yield mirtazapine having a mean particle size of less than 200 microns. Preferable the mirtazapine isolated has a mean particle diameter of less than 100 microns, more preferably less than 20 microns and most preferably about 10 microns.

According to another embodiment of the present invention, the micronized mirtazapine is made from dry mirtazapine.

According to another aspect, the present invention relates to a pharmaceutical composition comprising micronized mirtazapine.

It has been surprisingly and unexpectedly found that micronized mirtazapine has a superior dissolution profile compared to mirtazapine prior to micronization, including enhanced reproducibility of dissolution.

Surprisingly, micronized mirtazapine provides a form of mirtazapine in which the determination of an optimal dose is easier to determine and reproduce when compared to non-micronized mirtazapine.

### DESCRIPTION OF THE FIGURES

FIG. 1 is a graph of the dissolution data of mirtazapine before and after micronization.

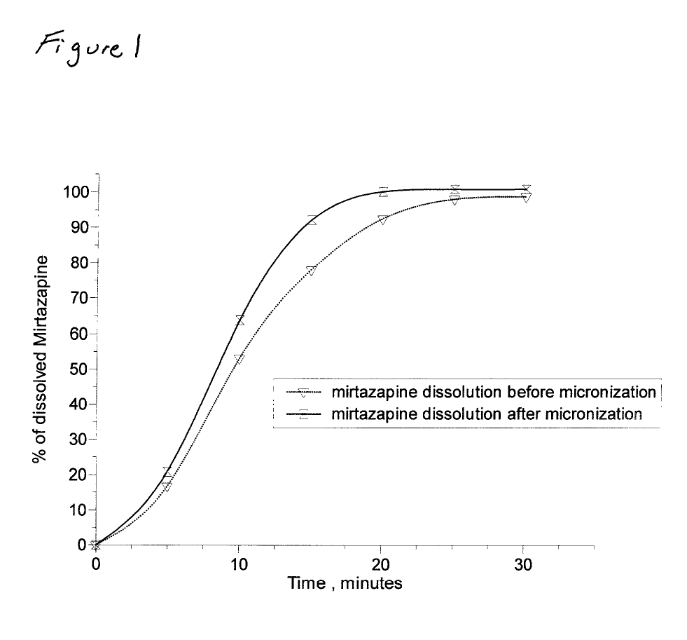

FIG. 2 is a graph of the percent of dissolved mirtazapine before and after micronization.

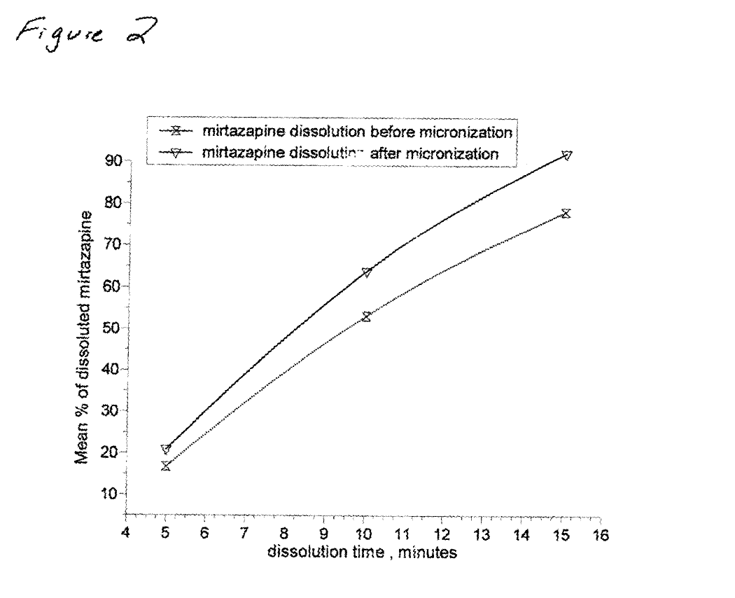

FIG. 3 is a graph of showing the dissolution time and standard deviations of the dissolution results before and after micronization.

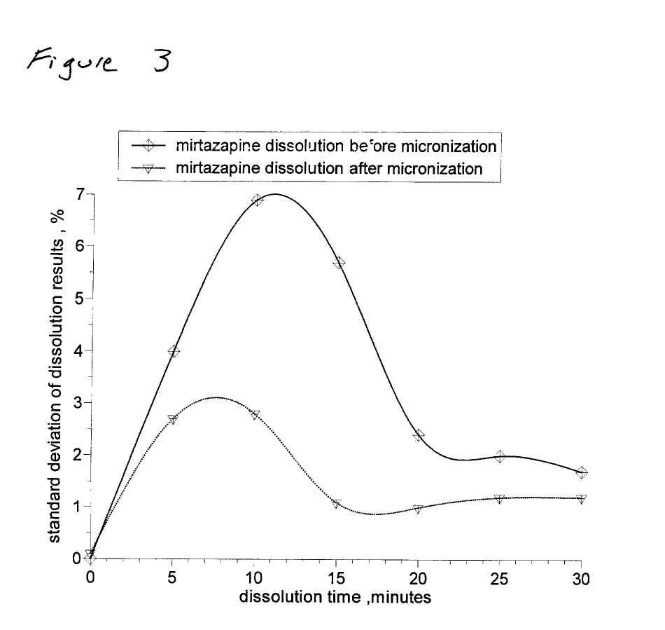

### DETAILED DESCRIPTION OF THE INVENTION

The present invention relates to mirtazapine formulations containing mirtazapine having relatively small particles, and corresponding relatively large surface area.

In one embodiment, the invention relates to mirtazapine and formulations containing mirtazapine having a mean particle diameter of less than 200 micrometer, preferably the mean particle diameter is less than 100 microns, more preferably the mean particle diameter is less than 20 microns, most preferably the mean particle size is between about 10 microns. In another embodiment of the present invention, mirtazapine has a mean particle diameter of between about 200 microns and 10 microns. In another embodiment of the present invention, mirtazapine has a mean length of about 4.2 microns, more preferably a mean length of 4.0 microns.

The present micronized mirtazapine has the unexpected results of possessing a more stable and reproducible dissolution profile. When compared to mirtazapine make by conventional methods, the present micronized mirtazapine unexpectedly demonstrated a more reproducible dissolution curve and a smaller standard of deviations. This is valuable improvement provides for more accurate dosing of mirtazapine.

According to another embodiment of the invention, the present invention relates to a process for preparing micronized mirtazapine. Mirtazapine prepared by methods known in the art is separated by sieves to produce mirtazapine wherein 50% has a mean particle diameter of below about 250 microns and about 80% has is below about 500 microns. The sieved mirtazapine is then micronized by methods known in the art, e.g., in a micronizer, to yield mirtazapine wherein 100% of the mirtazapine has a mean particle size of less than about 45 microns, preferably 99% of the mirtazapine has a mean particle size of less than about 45 microns, more preferably, 93% of the mirtazapine has a mean particle size of less than about 7.5 microns. more preferably the mirtazapine isolated has a mean particle diameter of less than 10 micron.

Micronized particles of mirtazapine can be obtained by the use of conventional micronizing techniques after sieving to provide mirtazapine wherein about 50% has a particle size less than 250 microns and about 80% has a particle sized below about 500 microns.

Mirtazapine may be micronized to the desired particle size range by conventional techniques, for example, using a ball mill, ultrasonic means, or using fluid energy attrition mills.

According to another embodiment of the present invention, the micronized mirtazapine is made from dry mirtazapine.

According to another aspect, the present invention relates to a pharmaceutical composition comprising micronized mirtazapine.

In accordance with the present invention, mirtazapine produced by the process of the present invention may be prepared as pharmaceutical compositions that are particularly useful for the treatment of depression. Such compositions comprise a therapeutically effective amount of mirtazapine with pharmaceutically acceptable carriers and/or excipients known to one of skill in the art.

### EXAMPLES

The present invention will now be further explained in the following example. However, the present invention should not be construed as limited thereby. One of ordinary skill in the art will understand how to vary the exemplified preparations to obtain the desired results.

It should be understood that some modification, alteration and substitution is anticipated and expected from those skilled in the art without departing from the teachings of the invention. Accordingly, it is appropriate that the following claims be construed broadly and in a manner consistent with the scope and spirit of the invention.

### Example 1

Pure dry Mirtazapine was micronized in a micronizer. The isolated product was micronized mirtazapine of a particle size of about 10 to 20 micrometer.

1. Mirtazapine having a mean particle diameter of less than 200 micrometer.

2. Mirtazapine of claim 1 wherein the mean particle diameter of less than 100 micrometer.

3. Mirtazapine of claim 2 wherein the mean particle diameter of less than about 20 micrometer.

4. Mirtazapine of claim 3 wherein the mean particle diameter is about 10 micrometer.

5. Mirtazapine of claim 1 wherein the mean particle size of less than 100 micrometers and greater than 10 micrometers.

6. A process for preparing micronized mirtazapine, which comprises micronizing mirtazapine.

7. The process according to claim 6 wherein the mirtazapine that is to be micronized is purified mirtazapine

8. The process according to claim 6 in which the mirtazapine that is to be micronized is dry mirtazapine.

9. The process of claim 6 wherein the mirtazapine that is to be micronized has a mean particle diameter of about 300 microns.

10. The process of claim 6 wherein the mirtazapine that is to be micronized has a mean particle diameter of about 250 microns.

11. Mirtazapine with a mean diameter of about 10 microns.

12. A pharmaceutical composition comprising a therapeutically effective amount micronized mirtazapine as claimed in any of claims 1 and 11.
